(19) United States
(12) Patent Application Publication
(10) Pub. No.: US 2024/0087711 A1
(43) Pub. Date: Mar. 14, 2024

(54) MEDICATION MANAGEMENT SYSTEM, MEDICATION MANAGEMENT METHOD, MEDICINE PACKAGING DEVICE, AND PACKAGED MEDICINE

(71) Applicant: Murata Manufacturing Co., Ltd., Nagaokakyo-shi (JP)

(72) Inventors: Takanori NAKAMURA, Nagaokakyo-shi (JP); Shohei MORIKAWA, Nagaokakyo-shi (JP); Kosuke HAMADA, Nagaokakyo-shi (JP); Hiroshi TAKEMURA, Nagaokakyo-shi (JP)

(21) Appl. No.: 18/518,961

(22) Filed: Nov. 25, 2023

Related U.S. Application Data
(63) Continuation of application No. PCT/JP22/10542, filed on Mar. 10, 2022.

(30) Foreign Application Priority Data
May 26, 2021 (JP) 2021-088240

(51) Int. Cl. G16H 20/13; G16H 10/60

(52) U.S. Cl. CPC G16H 20/13; G16H 10/60

(57) ABSTRACT

A medication management system, method, and related medicine packaging device and packaged medicine are provided. The exemplary system includes a medication device including a memory unit that stores ID information and an ID transmission unit that transmits the ID information stored in the memory unit. A processing device that has correspondence information in which the ID information is associated with prescription information of a plurality of medicines to be taken together with one medication device and personal information about a patient who takes the plurality of medicines. A detector detects the ID information transmitted from the ID transmission unit and transmits the ID information that has been detected to the processing device, which manages medication based on the ID information that has been received and the correspondence information.

### CROSS REFERENCE TO RELATED APPLICATIONS

This application is a continuation of International Application No. PCT/JP2022/010542, filed Mar. 10, 2022, which claims priority to Japanese Patent Application No. 2021-088240, filed May 26, 2021, the entire contents of each of which are hereby incorporated by reference in their entirety.

### TECHNICAL FIELD

The present invention relates to a medication management system, a medication management method, a medicine packaging device that places a medication device for managing medication into a container together with a plurality of medicines, and a packaged medicine in which the medication device for managing medication and the plurality of medicines is placed into the container.

### BACKGROUND

There has been a need for a system that can confirm whether or not a patient has taken a prescribed medicine.

As one of such systems, Japanese Patent Application Laid-open No. 2009-532119 (hereinafter “Patent Document 1”) discloses a system in which a signal transmission device having information such as a type, dose, and a serial number of a medicine is placed in a capsule together with the medicine, and when the capsule is taken, a signal is transmitted from the signal transmission device and received by an external reception device, thereby detecting that the medicine has been taken.

However, in the system described in Patent Document 1, when a plurality of medicines is taken at one time, it is necessary to associate each medicine with a signal transmission device and then place them in a capsule, which takes time and effort. In addition, since a plurality of signals corresponding to the number of medicines taken are transmitted from the signal transmission device, it becomes complicated to receive the plurality of signals by the external receiving device and manage medication.

### SUMMARY OF THE INVENTION

Accordingly, it is an object of the present invention to provide a technique for reducing the complexity of medication management when a plurality of medicines are taken at one time.

In an exemplary aspect, a medication management system is provided that includes a medication device including a memory unit that stores ID information and an ID transmission unit that transmits the ID information stored in the memory unit; a processing device that has correspondence information in which the ID information of the medication device is associated with prescription information of a plurality of medicines to be taken together with one of the medication devices and personal information about a patient who takes the plurality of medicines; and a detector configured to detect the ID information transmitted from the ID transmission unit when the one of the medication devices is taken by a patient, and to transmit the ID information that has been detected to the processing device. In this aspect, the processing device is configured to receive the ID information transmitted from the detector, and to manage medication based on the ID information that has been received and the correspondence information.

In another exemplary aspect, a medication management method is provided that includes causing a processing device to store correspondence information in which ID information of a medication device is associated with prescription information of a plurality of medicines to be taken together with one of the medication devices and personal information about a patient who takes the plurality of medicines; detecting, by a detector, the ID information transmitted from the one of the medication devices when the one of the medication devices is taken by the patient; transmitting the ID information detected by the detector to the processing device; receiving, by the processing device, the ID information transmitted from the detector; and managing, by the processing device, medication based on the ID information that has been received and the correspondence information.

In another exemplary aspect, a medicine packaging device is provided that includes a medicine accommodating unit configured to accommodate a plurality of medicines; a medication device accommodating unit configured to accommodate a medication device; an information processing unit configured to manage correspondence information in which ID information of the medication device is associated with prescription information of the plurality of medicines to be taken together with the medication device and personal information about a patient who takes the plurality of medicines; a control unit configured to issue a packaging instruction for placing the plurality of medicines to be taken by a patient at one time and one of the medication devices into one container based on the correspondence information; and a packaging unit configured to take out the plurality of medicines from the medicine accommodating unit according to the packaging instruction issued from the control unit, and to take out the one of the medication devices to be taken together with the plurality of medicines from the medication device accommodating unit to place the plurality of medicines and the one of the medication devices into the one container.

In yet another exemplary aspect, a packaged medicine is provided that includes a plurality of medicines to be taken by a patient at one time; a medication device including a memory unit configured to store ID information and an ID transmission unit configured to transmit the ID information stored in the memory unit; and a container containing the plurality of medicines and one of the medication devices.

According to the medication management system of an exemplary aspect described herein, the processing device has the correspondence information in which the plurality of medicines to be taken by a patient at one time and one medication device are associated with each other, and manages the medication on the basis of the ID information transmitted from the one medication device that has been taken and the correspondence information. Thus, it is not necessary to associate one medication device with one medicine, and the complexity of medication management is reduced.

According to the medication management method of an exemplary aspect described herein, the correspondence information in which the ID information of the medication device is associated with the prescription information of the plurality of medicines to be taken together with one medication device and the personal information about a patient, is stored in the processing device, and the ID information transmitted from the medication device when one medication device is taken by the patient, is detected by the detector, and then the ID information is transmitted to the processing device. Since the processing device is configured to manage medication based on the ID information transmitted from the detector and the correspondence information, it is not necessary to associate one medication device with one medicine, and the complexity of medication management is reduced.

Moreover, the medicine packaging device of an exemplary aspect described herein manages, by the information processing unit, the correspondence information in which the ID information of the medication device is associated with the prescription information of the plurality of medicines to be taken together with one medication device and the personal information about a patient, and places, by the packaging unit, the plurality of medicines and the medication device into the container according to the packaging instruction issued from the control unit based on the correspondence information, and thus, the complexity of medication management is reduced as compared with a configuration in which one medication device is associated with one medicine.

In addition, the packaged medicine of an exemplary aspect described herein has a configuration in which the plurality of medicines to be taken by a patient at one time and the one medication device are contained in the container. With such a configuration, it is not necessary to associate one medication device with one medicine, and thus, the complexity of medication management is reduced.

### BRIEF DESCRIPTION OF DRAWINGS

FIG. 1 is a block diagram schematically illustrating a configuration of a medication management system according to a first exemplary embodiment.

FIGS. 2(a) to 2(e) are diagrams illustrating various examples of a shape and a form of a medication device.

FIG. 3 is a diagram schematically illustrating a packaged medicine in which a plurality of medicines to be taken at one time and a medication device are contained in a container.

FIG. 4 is a diagram illustrating an example of correspondence information included in a processing device.

FIG. 5 is a flowchart illustrating a flow of processing of managing medication by the medication management system according to an exemplary embodiment.

FIG. 6 is a flowchart illustrating a flow of processing of managing medication by the medication management system in a case where a detector does not include a clock and the processing device includes a clock.

FIG. 7 is a block diagram schematically illustrating a configuration of a medicine packaging device according to an exemplary embodiment.

FIG. 8 is a diagram illustrating packaged medicines prepared according to a packaging instruction.

### DETAILED DESCRIPTION

Hereinafter, features of the present invention will be specifically described with reference to exemplary embodiments.

### First Exemplary Embodiment

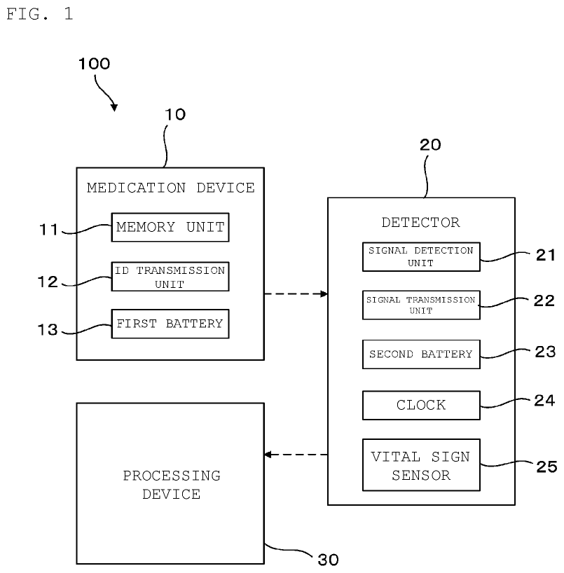

FIG. 1 is a block diagram schematically illustrating a configuration of a medication management system 100 according to a first exemplary embodiment. The medication management system 100 according to the first exemplary embodiment includes a medication device 10, a detector 20, and a processing device 30.

In the medication management system 100 according to the exemplary embodiment, when a plurality of medicines is taken at one time, one medication device 10 is associated with the plurality of medicines taken at one time. For purposes of this disclosure, the phrase “when a plurality of medicines is taken at one time” means that there is a plurality of medicines prescribed to be taken at the same time. Moreover, a person who takes a plurality of medicines together with the medication device 10 can be referred to as a “patient” in an exemplary aspect.

The medication device 10 is taken together with a plurality of medicines taken at one time. However, the medication device 10 and the plurality of medicines do not necessarily need to be taken at the same time and can be taken one by one within substantially the same time according to an exemplary aspect.

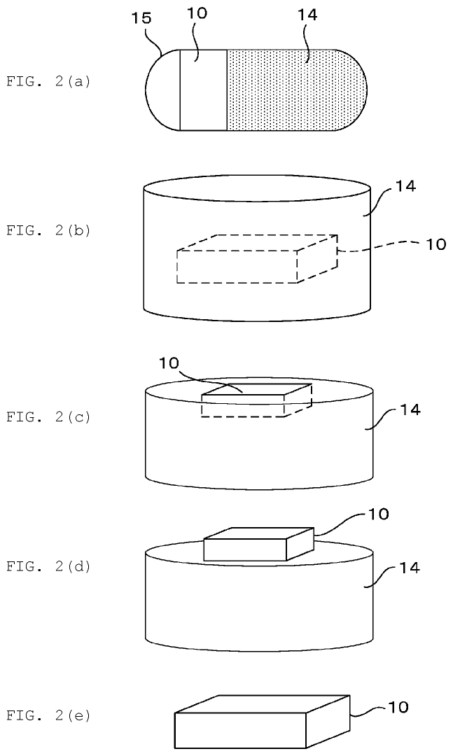

It is noted that the shape and form of the medication device 10 are not particularly limited. For example, as illustrated in FIG. 2(a), the medication device 10 is accommodated in a capsule 15 together with a placebo 14. As another form, the medication device 10 is configured as a set with the placebo 14 that is a tablet as illustrated in FIGS. 2(b) to 2(d). In the example illustrated in FIG. 2(b), the medication device 10 is provided in a manner buried inside the placebo 14 that is a tablet. In the example illustrated in FIG. 2(c), the medication device 10 is provided inside the placebo 14 in a manner that a part of the medication device 10 is exposed to a surface of the placebo 14. In the example illustrated in FIG. 2(c), a surface of the medication device 10 is exposed such that the surface of the medication device 10 and the surface of the placebo 14 form the same plane. In the example illustrated in FIG. 2(d), the medication device 10 is fixed to the surface of the placebo 14. It is also noted that the placebo 14 may be a medicine. As illustrated in FIG. 2(e), the medication device 10 may be used alone rather than in combination with the placebo 14. In that case, it is preferable that the surface of the medication device 10 is coated with a biocompatible material.

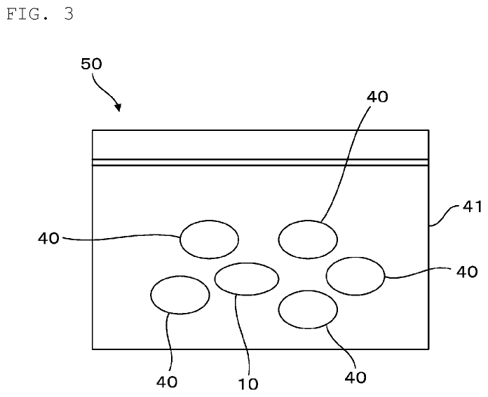

As illustrated in FIG. 3, the medication device 10 is delivered to a patient in a state of being placed in the container 41 together with a plurality of medicines 40 to be taken at one time. That is, the plurality of medicines 40 to be taken at one time and one medication device 10 are placed in the container 41. For purposes of this disclosure and according to an exemplary aspect, the plurality of medicines 40 to be taken by the patient at one time and one medication device 10 placed in the container 41 are referred to as a packaged medicine 50. That is, the packaged medicine 50 includes the medication device 10, the plurality of medicines 40 to be taken by the patient at one time, and the container 41 containing one medication device 10 and the plurality of medicines 40. The shape and form of the container 41 are not particularly limited, and for example, the container is an openable and closable transparent polypropylene bag or case according to an exemplary aspect.

The packaged medicine 50 is delivered to the patient by the number of doses. For example, in a case where the medicine is taken for one week after each meal in the morning, the daytime, and the night, a total of 21 packaged medicines 50 are delivered to the patient.

As further illustrated, the medication device 10 includes a memory unit 11, an ID transmission unit 12, and a first battery 13.

The memory unit 11 stores information (hereinafter, referred to as ID information) related to an ID for identifying the medication device 10. The ID information may be an ID itself of the medication device 10 or data that can identify the ID. For example, the ID information is input at a factory where the medication device 10 is manufactured and stored in the memory unit 11. In addition, the ID information need not be stored in the memory unit 11 at the time of manufacture, and the ID information may be input and stored in the memory unit 11 before the packaged medicine 50 is delivered to the patient at a pharmacy, a hospital, or the like.

The ID transmission unit 12 is configured to transmit the ID information stored in the memory unit 11. More specifically, when the medication device 10 is taken, the ID transmission unit 12 transmits the ID information stored in the memory unit 11. The ID transmission unit 12 is, for example, a piezoelectric element, and transmits the ID information as an ultrasonic signal which is a vibration signal. The frequency of the ultrasonic signal in this case is, for example, 20 kHz or more and 20 MHz or less. However, the signal when the ID transmission unit 12 transmits the ID information is not limited to the ultrasonic signal, and for example, a communication technology of each standard selected from Bluetooth®, radio frequency identification (RFID), near field communication (NFC), and the like, or a signal thereof may be used.

The first battery 13 supplies power to the ID transmission unit 12. The first battery 13 is, for example, a secondary battery or a gastric acid battery that generates electric power when gastric acid touches an electrode. For example, when a secondary battery is used as the first battery 13, it is preferable to use an all-solid-state battery having a solid electrolyte. In this case, for example, a solid electrolyte containing a NASICON-type crystal oxide can be used as the solid electrolyte. Since an electrolytic solution does not flow into a body by using an all-solid-state battery not containing an electrolytic solution as the secondary battery, safety is high. However, as long as safety can be secured, a secondary battery having an electrolytic solution can be used.

When the first battery 13 is a gastric acid battery, the medication device 10 is taken with a plurality of medicines, and electric power is supplied from the gastric acid battery to the ID transmission unit 12 in contact with gastric acid, whereby the ID information is transmitted. Furthermore, in a case where the first battery 13 is a secondary battery, for example, a signal is transmitted from the ID transmission unit 12 every predetermined time to receive a reflection signal, and when it is detected that the medication device 10 has been taken into the body on the basis of the strength of the received reflection signal, the ID information is transmitted from the ID transmission unit 12. That is, since the intensity of the reflection signal changes as the medication device 10 is taken into the body, it is possible to detect that the medication device 10 has been taken into the body based on the intensity of the reflection signal, and to transmit the ID information. However, the method of detecting that the medication device 10 has been taken into the body is not limited to the above-described method.

The detector 20 includes at least a signal detection unit 21, a signal transmission unit 22, and a second battery 23. The detector 20 according to the present embodiment further includes a clock 24 and a vital sign sensor 25.

In operation, the detector 20 can be held by a patient who takes the plurality of medicines 40 together with the medication device 10. However, for purposes of this disclosure, the term “held” means being managed by the patient and includes an aspect in which the patient not only wears the detector 20, but also leaves it in the house. For example, the detector 20 is a terminal device that is portable by a patient and can perform wireless communication. The detector 20 may include a position sensor for identifying a position. In order to reliably detect the ID information transmitted from the ID transmission unit 12 of the medication device 10, the detector 20 is preferably used in a state of being attached to the patient, particularly in a state of being attached to the body surface of the patient.

The signal detection unit 21 is configured to detect ID information transmitted from the ID transmission unit 12 of the medication device 10.

When the signal detection unit 21 detects the ID information of the medication device 10, the signal transmission unit 22 transmits the detected ID information to the processing device 30 described later by wireless communication. In the present embodiment, the signal transmission unit 22 acquires the time at which the signal detection unit 21 receives the ID information from the clock 24 to be described later, acquires information regarding a vital sign of the patient (hereinafter, referred to as vital sign information) from the vital sign sensor 25 to be described later, and transmits the time at which the ID information is received and the vital sign information to the processing device 30 together with the ID information of the medication device 10. The time at which the ID information is received can be regarded as the time at which the patient takes the plurality of medicines 40 together with the medication device 10. The wireless communication is, for example, communication using a data communication line using a radio wave of a mobile phone, an Internet line, Bluetooth®, or the like.

When the vital sign sensor 25 detects the vital sign information after the medicines 40 are taken, the signal transmission unit 22 is configured to transmit, to the processing device 30, the vital sign information after the medicines are taken. The vital sign information after the medicines are taken may be transmitted as desired.

Furthermore, when the detector 20 includes the position sensor, the signal transmission unit 22 can be configured to transmit the position information detected by the position sensor to the processing device 30 together with the ID information of the medication device 10. For example, when the patient is in a foreign country on a trip, by transmitting the position information detected by the position sensor to the processing device 30, the processing device 30 can recognize that the time at which the patient takes the medicine 40 is a local time in the foreign country.

The second battery 23 supplies power to the signal detection unit 21, the signal transmission unit 22, the clock 24, and the vital sign sensor 25. The second battery 23 is, for example, a secondary battery.

Furthermore, the clock 24 may be any component as long as the component can confirm a time. It is noted that the component that can confirm a time includes a component that manages a time inside the detector 20, that is, a component that manage a time only inside the detector 20 without displaying the time on a display or the like.

The vital sign sensor 25 is configured to detect vital sign information of a patient. The vital sign information is, for example, information for grasping a state of the patient's body such as a respiratory rate, a pulse, a blood pressure, and a body temperature. The vital sign sensor 25 detects at least vital sign information before and after a medicine is taken. The vital sign information before the medicine is taken may be, for example, information on a vital sign a predetermined time before the medicine is taken, such as one hour before the medicine is taken, or information on an average of vital signs detected a plurality of times before the medicine is taken. The vital sign information after the medicine is taken is, for example, vital sign information after a predetermined time after the medicine is taken, such as two hours after the medicine is taken. It is noted that the vital sign information may be acquired as desired, and the vital sign sensor 25 is not included in this case according to an alternative aspect.

The processing device 30 has correspondence information in which the ID information of the medication device 10 is associated with prescription information of a plurality of medicines 40 to be taken together with the medication device 10 and personal information about a patient who takes the plurality of medicines. The processing device 30 is, for example, a computer or a server on a cloud, and includes at least a CPU and a storage device. The processing device 30 can be configured to execute software applications to perform the algorithms described herein according to an exemplary aspect. Moreover, as described later, the processing device 30 may include a clock.

The prescription information is information regarding each of the medicines 40 to be prescribed to the patient and includes at least one of a type of the medicine 40, a dose of the medicine 40, and a prescription time of the medicine 40 according to an exemplary aspect. In order to better manage medication, the prescription information preferably includes all of the type of the medicine 40, the dose of the medicine 40, and the prescription time of the medicine 40. These pieces of prescription information are described in, for example, a prescription. The prescription information is, for example, based on a prescription, and is input in advance to the processing device 30 before a hospital or a pharmacy delivers the plurality of packaged medicines 50 to the patient.

The personal information is, for example, a number that can identify an individual, such as an Individual Number (a number uniquely assigned to each resident in a used country), a number described in a health insurance card, or a patient registration card number. For example, when the medication management system 100 is used in the United States, a social security number may be used as the personal information. Furthermore, the personal information may include at least one of age, gender, and medical history information. When the personal information includes at least one of age, gender, and medical history information, more complete medication management can be performed.

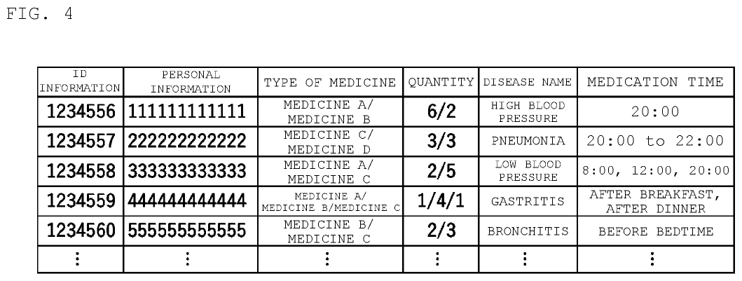

FIG. 4 is a diagram illustrating an example of correspondence information included in the processing device 30. In the example illustrated in FIG. 4, an Individual Number and a disease name as personal information, and a type of the medicine 40, a quantity of the medicine 40, and a medication time as prescription information are associated with ID information of the medication device 10. The correspondence information is input at a pharmacy or a hospital, for example, and is stored in the processing device 30. Referring to the correspondence information illustrated in FIG. 4, for example, it can be seen that a patient with Individual Number 111111111111 is scheduled to take the medication device 10 with ID 1234556, the medicines 40 scheduled to be taken are six tablets of medicine A and two tablets of medicine B, the patient's disease is high blood pressure, and the scheduled medication time of the medicines 40 is 20:00. The scheduled medication time is the time described in a prescription, and medicines may be taken before or after the scheduled medication time within a time range not affecting the prescription.

The processing device 30 is configured to receive the ID information of the medication device 10 transmitted from the detector 20 and further configured to manage medication based on the received ID information and the correspondence information. For example, when the processing device 30 receives ID information “1234556” illustrated in FIG. 4, it can be determined that the patient with Individual Number 111111111111 has taken six tablets of medicine A and two tablets of medicine B with reference to the correspondence information.

In the present embodiment, since the time at which the detector 20 receives the ID information is also transmitted from the detector 20, the processing device 30 can also be configured to grasp the time at which the patient with Individual Number 111111111111 takes the medicines 40. Therefore, in a case where the correspondence information includes the medication time, it is possible to determine whether or not the patient has taken the prescribed medicines 40 at a correct time. For example, in an example of the patient with Individual Number 111111111111, when the reception time of the ID information transmitted from the detector 20 to the processing device 30 is 8:00, it can be determined that the medicines have been erroneously taken at 8:00 although the medicines should have been taken at 20:00. It is also noted that when the processing device 30 determines that the patient has taken the medicines 40 by mistake, the processing device 30 can be configured to record that the patient has misjudged the medication time and issue an alert.

An example is now provided when there a plurality of the medicines 40 for one week are prescribed, and pieces of ID information of a plurality of the medication devices 10 for one week are different, but a plurality of the medicines 40 for the morning on the second day and a plurality of the medicines 40 for the morning on the third day are the same. In this case, the plurality of medicines 40 for the morning of the third day may be erroneously taken in the morning of the second day, but since the plurality of medicines 40 to be taken in the morning of the second day and the morning of the third day are the same, it is not determined that the medicines 40 are mistakenly taken, and the alert does not need to be issued.

Furthermore, in the present embodiment, since the vital sign information detected by the vital sign sensor 25, specifically, the vital sign information before and after taking the plurality of medicines 40 is transmitted from the detector 20, it is also possible to grasp a change in vital signs due to taking the plurality of medicines 40. A doctor can determine, for example, the type, quantity, and the like of each medicine 40 to be prescribed next time on the basis of the change in vital signs due to the patient taking the plurality of medicines 40.

It is noted that when the same medicine 40 is repeatedly taken, for example, as in a case where the medicine is taken after every breakfast, a plurality of the medication devices 10 having the same ID information may be prepared and associated with a plurality of the medicines 40 to be taken. As an example, in a case where the medicine 40 for one week is prescribed to the patient with Individual Number 111111111111 illustrated in FIG. 4, seven medication devices 10 with ID 1234556 are prepared, and each medication device 10 is associated with six tablets of medicine A and two tablets of medicine B. In that case, ID information 1234556 should be transmitted from the detector 20 to the processing device 30 around 20:00 every day if the patient takes the medicines correctly. In addition, for example, when the patient erroneously takes two sets of six tablets of medicine A and two tablets of medicine B at 20:00, ID information 1234556 is transmitted from the detector 20 to the processing device 30 twice. The processing device 30 can determine that the patient has erroneously taken the medicines twice by receiving ID information 1234556 twice a day.

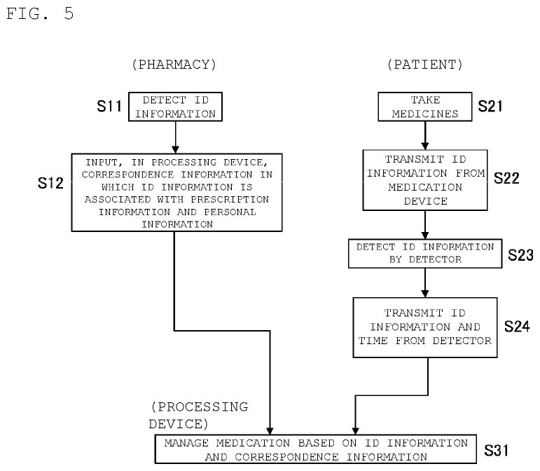

Hereinafter, a flow of processing of managing medication by the medication management system 100 according to the exemplary embodiment will be described with reference to the flowchart illustrated in FIG. 5. In particular, a description will be given assuming that, in a pharmacy, the packaged medicine 50 including the container 41 containing a plurality of the medicines 40 to be taken at one time and the medication device 10 is delivered to a patient. However, it should be appreciated that the place where the packaged medicine 50 is delivered to the patient is not limited to the pharmacy, and may be a hospital, for example.

The processing performed in steps S11 and S12 in FIG. 5 is processing performed in a pharmacy. The processing performed in steps S21 to S24 in FIG. 5 is processing performed on the patient side. The processing in step S31 in FIG. 5 is processing performed by the processing device 30.

The medication device 10 is manufactured in a factory and delivered to a pharmacy, for example. According to an exemplary aspect, it is assumed that the medication device 10 manufactured in the factory already includes ID information. In the pharmacy, ID information of the medication device 10 is detected using a predetermined ID detector (step S11).

Subsequently, in the pharmacy, correspondence information in which the ID information of the medication device 10 is associated with prescription information of the plurality of medicines 40 to be taken together with the medication device 10 and personal information about the patient who takes the plurality of medicines is stored in the processing device 30 (step S12). For example, correspondence information is input to a computer installed in the pharmacy, and the correspondence information is transmitted to the processing device 30 via the Internet. As a result, the correspondence information is stored in the processing device 30.

Here, as the prescription information, for example, information described in a prescription is input. As the personal information, information described in the prescription may be input, or information provided from the patient (for example, information from a medical interview form and a health insurance card) may be input. In addition, when the packaged medicine 50 is delivered to the patient for the second time or more, and the personal information has already been input, the input personal information may be used. As described above, the correspondence information in which one medication device 10 is associated with the plurality of medicines 40 taken by the patient at one time is managed. The associated one medication device 10 and the plurality of medicines 40 are accommodated in the container 41 and delivered to the patient as the packaged medicine 50.

When a medication time comes, the patient who has received the packaged medicine 50 at the pharmacy takes out the plurality of medicines 40 and the medication device 10 from the container 41 and takes the plurality of medicines 40 and the medication device 10 (step S21). It should be appreciated that there are no particular limitations on the medication method and the medicines and the medication device may be taken at once, or may be taken one by one within substantially the same time period. When the medication device 10 is taken into the body, ID information is transmitted from the ID transmission unit 12 (step S22).

The signal detection unit 21 of the detector 20 is configured to detect the ID information transmitted from the ID transmission unit 12 of the medication device 10 (step S23).

The signal transmission unit 22 of the detector 20 is configured to acquire the time at which the signal detection unit 21 detects the ID information and further configured to transmit the ID information detected by the signal detection unit 21 and the time at which the signal detection unit 21 detects the ID information to the processing device 30 by wireless communication (step S24). Moreover, in an exemplary aspect of the present embodiment, the vital sign information detected by the vital sign sensor 25 can also be transmitted together from the signal transmission unit 22 to the processing device 30.

The processing device 30 is configured to receive the ID information of the medication device 10 transmitted from the detector 20 and further configured to manage medication based on the received ID information and the correspondence information stored in advance (step S31).

Although the detector 20 has been described as including the clock 24 for detecting the time at which the signal detection unit 21 detects the ID information, the clock 24 need not be included, and the processing device 30 may include a clock instead. In this case, the time at which the processing device 30 receives the ID information transmitted from the detector 20 can be regarded as the time at which the patient takes the plurality of medicines 40. It is also noted that there is a difference between the time at which the patient takes the medicines 40 and the time at which the processing device 30 receives the ID information transmitted from the detector 20, but there is no particular problem since an accurate time is not necessarily required in managing the medication.

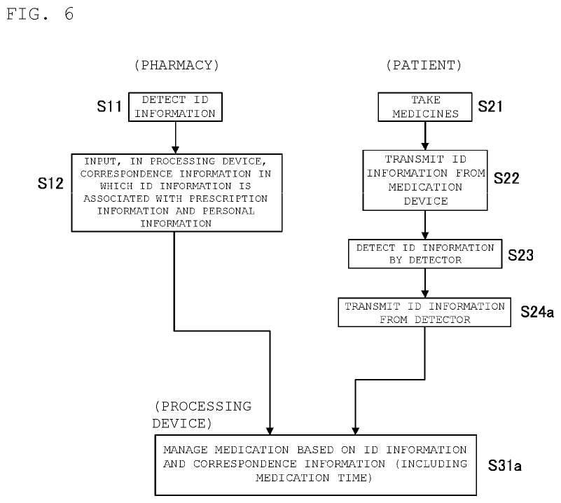

FIG. 6 is a flowchart illustrating a flow of processing in a case where the detector 20 does not include the clock 24 and the processing device 30 includes a clock. The flowchart illustrated in FIG. 6 is different from the flowchart illustrated in FIG. 5 in processing in step S24a and step S31a. Therefore, only the processing of step S24a and step S31a will be described below.

The signal transmission unit 22 of the detector 20 transmits the ID information detected by the signal detection unit 21 to the processing device 30 by wireless communication (step S24a). In the present embodiment, the vital sign information detected by the vital sign sensor 25 is also transmitted together from the signal transmission unit 22 to the processing device 30.

The processing device 30 is configured to receive the ID information of the medication device 10 transmitted from the detector 20 and further configured to manage medication based on the received ID information and the correspondence information stored in advance (step S31a). At this time, the processing device 30 is configured to acquire the time at which the ID information is received from the clock included in the processing device 30, regards the acquired time as the time at which the patient takes the plurality of medicines 40, and manages the medication.

The above-described medication management method will be summarized. The medication management method in an exemplary embodiment includes causing the processing device 30 to store the correspondence information in which the ID information of the medication device 10 is associated with the prescription information of the plurality of medicines 40 to be taken together with one medication device 10 and the personal information about a patient who takes the plurality of medicines; detecting, by the detector 20, the ID information transmitted from the medication device 10 when the one medication device 10 is taken by a patient; transmitting the ID information detected by the detector 20 to the processing device 30; receiving, by the processing device 30, the ID information transmitted from the detector 20; and managing, by the processing device 30, the medication based on the received ID information and the correspondence information.

In addition, the medication management method in an exemplary embodiment may further include acquiring a time at which the ID information is detected when the detector 20 detects the ID information of the medication device 10, and in the transmitting of the ID information to the processing device 30 by the detector 20, the time at which the ID information is detected may be transmitted to the processing device 30 together with the detected ID information.

According to the medication management system 100 in the exemplary embodiment, the processing device 30 has the correspondence information in which the plurality of medicines 40 to be taken by a patient at one time and one medication device 10 are associated with each other, and manages the medication on the basis of the ID information transmitted from the medication device 10 that has been taken and the correspondence information, and thus, it is not necessary to associate one medication device 10 with one medicine 40. Therefore, as compared with a conventional system in which one medication device is associated with one medicine, the number of medication devices 10 to be prepared can be reduced, and the number of signals to be processed is reduced, so that medication management is facilitated.

In addition, since the detector 20 includes the clock 24 and is configured to transmit the time at which the ID information is detected together with the ID information of the detected medication device 10 to the processing device 30, it is possible to grasp not only the presence or absence of medication, but also the time at which the patient takes the medicines 40, and thus, the medication can be more accurately managed.

In addition, since the detector 20 includes the vital sign sensor 25 and is configured to transmit a vital sign of the patient together with the ID information of the detected medication device 10 to the processing device 30, it is possible to grasp not only the presence or absence of medication but also a change in vital sign due to medication, and thus, the medication can be more accurately managed.

Furthermore, when the processing device 30 instead of the detector 20 includes a clock, and the time at which the plurality of medicines 40 are taken is managed on the basis of the time at which the ID information of the medication device 10 transmitted from the detector 20 is received, it is possible to grasp the time at which the patient takes the medicines 40 while simplifying the configuration of the detector 20.

(Medicine Packaging Device)

As described above, one medication device 10 is associated with the plurality of medicines 40 to be taken at one time and placed in the container 41. The one medication device 10 and the plurality of medicines 40 associated with the medication device 10 are placed in the container 41 by, for example, a medicine packaging device and delivered to a patient as the packaged medicine 50. The medicine packaging device is disposed in, for example, a pharmacy or a hospital.

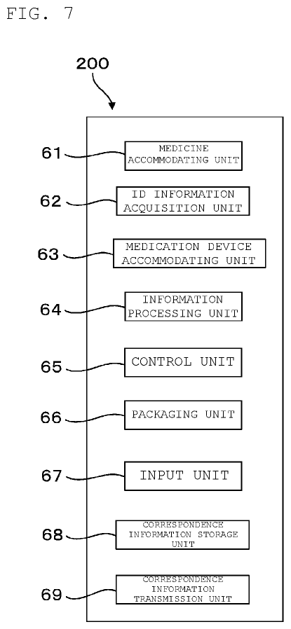

FIG. 7 is a block diagram schematically illustrating a configuration of a medicine packaging device 200 according to an exemplary embodiment. The medicine packaging device 200 includes a medicine accommodating unit 61, an ID information acquisition unit 62, a medication device accommodating unit 63, an information processing unit 64, a control unit 65, and a packaging unit 66. The medicine packaging device 200 according to the present embodiment further includes an input unit 67, a correspondence information storage unit 68, and a correspondence information transmission unit 69.

The medicine accommodating unit 61 is configured to accommodate the medicines 40. For example, the medicine accommodating unit 61 includes a plurality of cassettes for accommodating the medicines 40 for different types and accommodates the medicines 40 such that the medicines 40 of the same type are placed in the same cassette.

The ID information acquisition unit 62 is configured to acquire ID information of the medication device 10.

The medication device accommodating unit 63 accommodates the plurality of medication devices 10 the ID information of which is acquired by the ID information acquisition unit 62. It is preferable to charge the first battery 13 of the medication device 10 used by the medicine packaging device 200 on the day among the plurality of medication devices 10.

The input unit 67 is configured for inputting prescription information and personal information, and is, for example, a computer.

The information processing unit 64 is configured to associate the prescription information and the personal information input by the input unit 67 with the ID information of the medication device 10. That is, on the basis of the prescription information and the personal information, the plurality of medicines 40 taken by a patient at one time with the ID information of one medication device 10, and correspondence information is managed. Note that correspondence information managed (e.g., stored) by the processing device 30 may be captured into the information processing unit 64 by a predetermined method and managed and used as the correspondence information. In this case, the medicine packaging device 200 may further include a communication unit for transmitting and receiving information (e.g., data) to and from the processing device 30.

Based on the correspondence information managed by the information processing unit 64, the control unit 65 issues, to the packaging unit 66, a packaging instruction for placing the plurality of medicines 40 to be taken by the patient at one time and one medication device 10 into one container 41. As an example, a packaging instruction for placing the medication device 10 with ID information 1234556 illustrated in FIG. 4, and six tablets of medicine A and two tablets of medicine B in one container 41, is issued.

The packaging unit 66 is configured to take out the plurality of medicines 40 from the medicine accommodating unit 61 according to the packaging instruction issued by the control unit 65 and further configured to take out one medication device 10 to be taken together with the plurality of medicines 40 from the medication device accommodating unit 63 and to place the medication device 10 into the container 41. Thereby, the packaged medicine 50 is obtained. When the number of prescription days is seven days, seven packaged medicines 50 are obtained. In addition, in a case where the medicines are taken three times a day in the morning, the daytime, and the night, and the number of prescription days is seven days, a total of 21 packaged medicines 50 are obtained. Since the packaged medicine 50 allows the medication of the plurality of medicines 40 taken by the patient at one time to be managed with one medication device 10, it is possible to reduce the complexity of the medication management as compared with the conventional configuration in which the medication of one medicine is managed with one medication device.

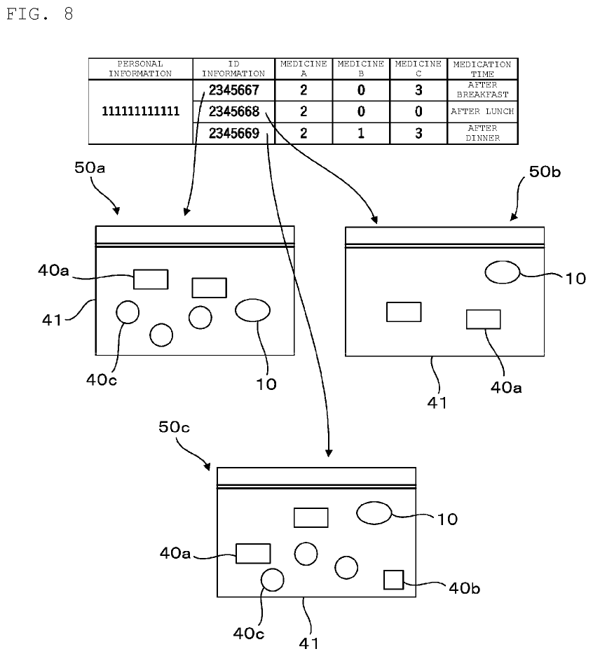

FIG. 8 is a diagram illustrating the packaged medicines 50 prepared according to a packaging instruction. In the example illustrated in FIG. 8, since the patient with Individual Number 111111111111, which is the personal information, takes the medicines after breakfast, a packaged medicine 50a is prepared according to a packaging instruction to place the medication device 10 with ID 2345667, two tablets of a medicine A 40a, and three tablets of a medicine C 40c into the container 41. In addition, in order to take the medicines after lunch, a packaged medicine 50b is prepared according to a packaging instruction for placing the medication device 10 with ID 2345668 and two tablets of the medicine A 40a in the container 41. In addition, in order to take the medicines after dinner, a packaged medicine 50c is prepared according to a packaging instruction for placing the medication device 10 with ID 2345669 and two tablets of the medicine A 40a, one tablet of a medicine B 40b, and three tablets of the medicine C 40c in the container 41.

The correspondence information storage unit 68 stores correspondence information managed by the information processing unit 64.

The correspondence information transmission unit 69 transmits the correspondence information stored in the correspondence information storage unit 68 to the processing device 30.

According to the medicine packaging device 200 of the present embodiment, the information processing unit 64 is configured to manage correspondence information in which ID information of the medication device 10 is associated with prescription information of the plurality of medicines 40 to be taken together with one medication device 10 and personal information about a patient, and the control unit 65 is configured to issue a packaging instruction on the basis of the correspondence information. Since the packaging unit 66 places the plurality of medicines 40 and one medication device 10 into the container 41 according to the packaging instruction, the complexity of medication management is reduced as compared with a configuration in which one medication device 10 is associated with one medicine 40.

In general, it should be appreciated that the present invention is not limited to the above exemplary embodiment, and various applications and modifications can be made within the scope of the present invention as would be appreciated to one skilled in the art.

### DESCRIPTION OF REFERENCE SYMBOLS

10: Medication device
11: Memory unit
12: ID transmission unit
13: First battery
14: Placebo
15: Capsule
20: Detector
21: Signal detection unit
22: Signal transmission unit
23: Second battery
24: Clock
25: Vital sign sensor
30: Processing device
40, 40a, 40b, 40c: Medicine
41: Container
50, 50a, 50b, 50c: Packaged medicine
61: Medicine accommodating unit
62: ID information acquisition unit
63: Medication device accommodating unit
64: Information processing unit
65: Control unit
66: Packaging unit
67: Input unit
68: Correspondence information storage unit
69: Correspondence information transmission unit
100: Medication management system
200: Medicine packaging device

1. A medication management system comprising:
at least one medication device including a memory unit configured to store ID information and an ID transmission unit configured to transmit the stored ID information;
a processing device having correspondence information that associates the ID information of the at least one medication device with prescription information of a plurality of medicines to be taken together with one of the at least one medication device and further has personal information relating to a patient prescribed to take the plurality of medicines; and
a detector configured to detect the ID information transmitted from the ID transmission unit when the one of the at least one medication device is taken by the patient, and to transmit the ID information that has been detected to the processing device,
wherein the processing device is further configured to receive the ID information transmitted from the detector, and to manage medication based on the received ID information and the correspondence information.

2. The medication management system according to claim 1, wherein the detector is further configured to transmit, to the processing device, a time at which the ID information is detected together with the detected ID information.

3. The medication management system according to claim 1, wherein the detector includes a vital sign sensor configured to detect a vital sign of the patient, and to transmit the vital sign to the processing device together with the detected ID information.

4. The medication management system according to claim 1, wherein the processing device is further configured to manage a time at which the plurality of medicines are taken together with the medication device based on a time at which the ID information transmitted from the detector is received.

5. The medication management system according to claim 1, wherein the prescription information includes at least one of a type of each of the plurality of medicines, a dose of each of the plurality of medicines, and a prescription time of each of the plurality of medicines.

6. The medication management system according to claim 1, wherein the personal information of the patient includes at least one of age, gender, and medical history information.

7. The medication management system according to claim 1, wherein the detector is a terminal device that is portable by a patient and configured for wireless communication.

8. The medication management system according to claim 7, wherein the detector includes a position sensor configured to specify a position of the terminal device.

9. A medication management method comprising:
storing, by a processing device, correspondence information in which ID information of at least one medication device is associated with prescription information of a plurality of medicines to be taken together with one of the at least one medication device and personal information relating to a patient prescribed to take the plurality of medicines;
detecting, by a detector, the ID information transmitted from the one of the at least one medication device when the one of the at least one medication device is taken by the patient;
transmitting the detected ID information to the processing device; and
receiving, by the processing device, the detected ID information, and managing, by the processing device, medication based on the received ID information and the correspondence information.

10. The medication management method according to claim 9, further comprising acquiring a time at which the ID information is detected by the detector.

11. The medication management method according to claim 10, wherein the transmitting of the ID information to the processing device further comprises transmitting the time at which the ID information is detected together with the detected ID information.

12. The medication management method according to claim 9, further comprising:
detecting, by a vital sign sensor, a vital sign of the patient; and
transmitting the vital sign to the processing device together with the detected ID information.

13. The medication management method according to claim 9, further comprising managing, by the processing device, a time at which the plurality of medicines are taken together with the medication device based on a time at which the ID information transmitted from the detector is received.

14. The medication management method according to claim 9,
wherein the prescription information includes at least one of a type of each of the plurality of medicines, a dose of each of the plurality of medicines, and a prescription time of each of the plurality of medicines, and
wherein the personal information of the patient includes at least one of age, gender, and medical history information.

15. A medicine packaging device comprising:
a medicine accommodating unit configured to accommodate a plurality of medicines;
a medication device accommodating unit configured to accommodate at least one medication device;
an information processing unit configured to manage correspondence information in which ID information of the at least one medication device is associated with prescription information of the plurality of medicines to be taken together with the at least one medication device and personal information relating to a patient prescribed to take the plurality of medicines;
a control unit configured to issue a packaging instruction for placing the plurality of medicines to be taken by the patient at one time and the at least one medication device into one container based on the correspondence information; and
a packaging unit configured to take out the plurality of medicines from the medicine accommodating unit based on the packaging instruction issued from the control unit, and to take out the at least one medication device to be taken together with the plurality of medicines from the medication device accommodating unit to place the plurality of medicines and the at least one medication device into the one container.

16. The medicine packaging device according to claim 15, further comprising:
an ID information acquisition unit configured to acquire the ID information; and
a correspondence information storage unit configured to store the correspondence information.

17. The medicine packaging device according to claim 16, further comprising a correspondence information transmission unit configured to transmit the correspondence information stored in the correspondence information storage unit to a processing device that manages medication.

18. The medicine packaging device according to claim 17, further comprising an input unit configured to receive the prescription information and the personal information.

19. A packaged medicine comprising:
a plurality of medicines to be taken by a patient at one time;
at least one medication device including a memory unit configured to store ID information and an ID transmission unit configured to transmit the ID information stored in the memory unit; and
a container containing the plurality of medicines and one of the at least one medication device.

20. The packaged medicine according to claim 19, wherein the medication device includes a battery configured to supply power to the ID transmission unit.
